(19) United States
(12) Patent Application Publication
(10) Pub. No.: US 2005/0175976 A1
(43) Pub. Date: Aug. 11, 2005

(54) Meiosis activating sterol augments implantation rate

(76) Inventors: Claus Yding Andersen, Copenhagen O (DK); Anne Grete Byskov, Gentofte (DK)

Correspondence Address: Reza Green;Novo Nordisk Pharmaceuticals, Inc., 100 College Road West, Princeton, NJ, 08540, US

(21) Appl. No.: 10/626,053

(22) Filed: Jul. 24, 2003

Related U.S. Application Data
(63) Continuation of application No. 09/929,800, filed on Aug. 14, 2001, now abandoned.
Application No. 09/929,800 is a continuation of application No. PCT/DK00/00080, filed on Feb. 25, 2000.

(30) Foreign Application Priority Data
Feb. 26, 1999 (DK) PA 1999 00273

(51) Int. Cl. A01K 67/027

(52) U.S. Cl. 435/2; 800/18; 800/21

(57) ABSTRACT

The present invention relates to the use of a new principle for improving the viability and pregnancy potential of oocytes and pre-embryos obtained in connection with in vitro fertilisation and pre-embryo transfer treatment. More specifically, improvement by raising the content of Meiosis Activating Sterols (MAS) in the medium where the in vitro fertilisation takes place. This is achieved by exposing and culturing one or more oocytes with spermatozoa in a culture medium comprising at least one meiosis activating sterol (MAS), MAS analogue, and/or an additive or additives capable of endogenous stimulation of the accumulation of at least one MAS. Preferred additives are FSH and EGF.

### FIELD OF INVENTION

The present invention relates to the use of a new principle for improving the viability and pregnancy potential of oocytes and pre-embryos obtained in connection with in vitro fertilisation and pre-embryo transfer treatment. More specifically, the application relates to improvement of the pregnancy potential of oocytes and pre-embryos by raising the content of Meiosis Activating Sterols (MAS) in the medium where at least the in vitro fertilisation takes place and preferably also in the medium wherein the oocytes are cultured prior to in vitro fertilisation.

### BACKGROUND OF THE INVENTION

Couples seeking infertility treatment, will, almost without exception, be subject to fertilisation in vitro, wherein the meeting between an oocyte and a spermatozoa takes place in vitro. The woman is most commonly receiving treatment with exogenous hormones in order to regulate and stimulate the ovaries to develop more than the usual one preovulatory follicle which is seen during the natural menstrual cycle. Part of the treatment involves retrieving the oocytes from the preouvlatory follicles of the ovaries in order for the oocytes to be matured and/or fertilised in vitro. After fertilisation and pre-embryo development, one to three pre-embryos are replaced to the woman's uterus, and she thus has the possibility of becoming pregnant and carrying her own child. This is now an established treatment, which has been performed on a large scale for more than 20 years.

Although the likelihood of achieving a pregnancy has increased over the years, the frequency with which the retrieved oocytes undergo fertilisation and pre-embryo development has remained remarkably constant around 60-70%. However, only around 15 to 25 percent of the replaced pre-embryos implant and develop into offspring. Therefore, only a fraction of the retrieved oocytes will result in a conceptus. The reason why only such a small fraction of the in vitro developing pre-embryos possesses the capacity to implant is largely unknown, but several explanations have been suggested (Salha et al., 1998): the follicles/oocytes lack sufficient maturation; the follicle/oocyte has been exposed to sub-optimal concentrations of gonadotropins; the culture conditions of the oocyte may not reflect the conditions of the luminal fluid within the female reproductive tract; the oocyte may suffer from sub-optimal culture conditions.

In its natural surroundings, the oocyte is expelled from the follicle in a sea of follicular fluid which surrounds the oocyte during the first time in the fallopian tube. The follicular fluid is mixed with secretions from the fallopian tube, defining the environment in which fertilisation takes place. The follicular fluid contains a number of substances which are believed to enhance oocyte nuclear and oocyte cytoplasmatic maturation. Recent reports show that particularly follicle-stimulating hormone (FSH) and epidermal growth factor (EGF) participate in these maturation processes. They are both present in follicular fluid and the fallopian tube synthesise EGF particularly around ovulation (Morishige et al., 1993).

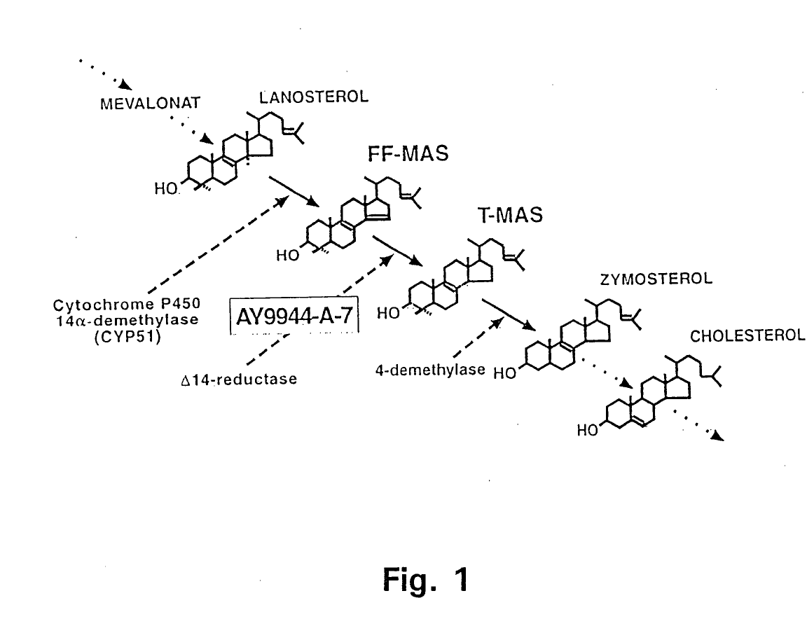

Mammalian oocytes are arrested in the prophase of the first meiotic division characterised by the presence of the nuclear membrane, and/or germinal vesicle (GV). When the oocyte resume meiosis it is visualised by the germinal vesicle break down (GVBD), also termed oocyte maturation. In the follicle, the oocytes stay in the GV stage as the result of the maturation inhibiting effect of hypoxanthine (HX) and other purines present in follicular fluid. Oocytes in culture also remain in the GV stage when cultured in the presence of physiological concentrations of HX and resume meiosis if HX is removed. The inhibitory effect of HX on the resumption of meiosis in cultured mouse cumulus enclosed oocytes, as well as cumulus deprived, naked oocytes, can be overcome by adding meiosis activating sterols, e.g. FF-MAS (4,4-dimethyl-5α-cholesta-8,14,24-triene-3β-ol) or T-MAS (4,4-dimethyl-5α-cholest-8,24-diene-3β-ol). FF-MAS was isolated and characterised from human follicular fluid and T-MAS from bull testis (Byskov et al., 1995). The sterols are intermediates in the cholesterol biosynthetic pathway and are immediate products of lanosterol (Schroepfer et al., 1972) (FIG. 1).

The synthesis of FF-MAS from lanosterol is catalysed by cytochrome P450 lanosterol 14α-demethylase (P45014DM) encoded by the CYP51 gene. FF-MAS is converted to T-MAS by the activity of sterol 14-reductase (Δ14R). The drug AY9944-A-7 (AY), which in the 1950s was used to lower plasma cholesterol, selectively inhibits the activity of Δ14R. The chemical structure of AY is totally unrelated to sterols and the intermediates in the cholesterol biosynthesis (for review: Mercer, 1993). Several studies have shown that cholesterol producing cells accumulate 4,4-dimethyl-5α-cholesta-8,14,24-triene-3β-ol (i.e. FF-MAS) when cultured in the presence of AY. It was shown that AY (0.1 μM) acts as a competitive inhibitor of the Δ14R in cultured rat hepatocytes.

Physiological concentrations of FSH have been shown to promote oocyte maturation in mice in vitro, and also in oocytes in germinal vesicle stage of human and monkey oocytes (Schramm and Bavister 1995). It has been shown that FSH stimulates cumulus cells to synthesise substances which positively promote maturation (Byskov et al., 1997). However, a recent study indicated that FSH reduced the rate of triploidy in a group of pre-embryos grown in the presence of FSH, suggesting a specific role of FSH in the final stages of meiosis (Merriman et al., 1998). Furthermore, by replacing these pre-embryos to pseudopregnant mice it was shown that the number of implantations were enhanced in the group with FSH present in the oocyte maturation medium compared to the control medium and almost similar to that of in vivo matured oocytes.

EGF has been shown to enhance oocyte maturation in a number of species like the cow, pig, mouse and rat, and one study found that FSH seems to up-regulate the expression of EGF receptors in rat granulosa cells (Maruo et al. 1993). EGF promotes the ability of mouse two-cells to develop into blastocysts (Morishige et al., 1993). In human oocytes results are less clear. A 6 h exposure of EGF in concentrations of 1 and 10 ng/ml was not reflected in augmented fertilisation rates of oocytes obtained from an IVF program (Gòmez et al. 1993), whereas Goud et al. (1998) found that EGF (2 ng/ml) enhanced the number of germinal vesicle oocytes reaching metaphase II (MII) after a 30 h culture and concluded that EGF improved the nuclear and cytoplasmatic maturation of human oocytes in vitro.

During the established treatment, the in vitro fertilisation takes place in a basal medium (Salha et al., 1998; Trounson and Gardner 1993). In such basal medium, the oocytes are devoid the natural environment of the follicular fluid. Thus, e.g. Merriman et al. 1998 wash the oocytes, cultured in a medium with FSH and EGF prior to transfer to a basal medium wherein in vitro fertilisation takes place.

### DETAILED DISCLOSURE OF THE INVENTION

One problem solved by the present invention is the very low rate of implantation of the in vitro created pre-embryos. This is achieved by exposing and culturing one or more oocytes with spermatozoa in a culture medium, the culture medium comprising at least one meiosis activating sterol (MAS), a MAS being any sterol in the metabolic pathway between lanosterol and cholesterol, a MAS analogue, and/or an additive or additives capable of endogenous stimulation of the accumulation of at least one MAS.

The culture medium wherein the actual in vitro fertilisation takes place subject to the present invention is based on the finding presented in the examples. Thus, in example 2 it is demonstrated that the implantation rate in humans is significantly higher in the group of oocytes/pre-embryos exposed to a medium containing FSH and EGF during in vitro fertilisation compared to those exposed to the control medium. Furthermore, the presence of FSH and EGF causes an accumulation of FF-MAS and T-MAS in the culture medium in which oocytes have grown as compared to the control medium. In example 1 it is demonstrated that the presence of FSH and EGF in cultures of cumulus enclosed oocytes in mice causes the accumulation of endogenous produced FF-MAS and T-MAS by attenuating the conversion of T-MAS to cholesterol.

Examples 1 and 2 not only show that the implantation rate is markedly increased when the in vitro fertilisation is performed in a medium with EGF and FSH in concentrations around those observed in vivo, but also substantiate that the underlying mechanism is the endogenous production of MAS and the other examples underline these findings. Example 3 demonstrates that FSH and EGF enhance oocyte maturation of mouse oocytes in vitro and that a combination of FSH and EGF seems to enhance the effect. Example 5 shows that an inhibitor of sterol 14-reductase (AY9944-A-7) stimulates oocyte maturation of cumulus enclosed mouse oocytes in a dose-dependent way, and that the presence of the AY compound in cultures of cumulus enclosed mice oocytes causes the accumulation of endogenous produced FF-MAS and T-MAS by attenuating the conversion of T-MAS to cholesterol. These findings demonstrate for the first time that cumulus oocyte complexes of mice are able to produce and accumulate FF-MAS in the presence of AY. Similar results are obtained with a cholesterol biosynthesis inhibitor, Amphotericin B, in example 6.

Therefore, enhancing the endogenous production of MAS represents a new mechanism to augment the pregnancy potential of pre-embryos fertilised in vitro.

Thus, one aspect of the invention relates to a method for in vitro fertilisation comprising the step of exposing and culturing one or more MII oocytes with spermatozoa in a culture medium, the culture medium comprising at least one meiosis activating sterol (MAS), a MAS being any sterol in the metabolic pathway between lanosterol and cholesterol, a MAS analogue; and/or an additive or additives capable of endogenous stimulation of the accumulation of at least one MAS. It is preferred that the in vitro fertilisation is in vitro fertilisation of human oocytes.

The in vitro fertilisation procedure is preferably performed as follows: the intact cumulus-oocyte complexes are localised in the follicular aspirate and transferred to the culture medium comprising at least MAS, a MAS analogue, and/or an additive or additives capable of endogenous stimulation of the accumulation of at least one MAS. After a few hours, purified spermatozoa are added to the cumulus-enclosed-oocyte and in vitro fertilisation takes place, which the next day (after 16-24 hour of culture) can be visualised by the presence of two pronuclei in the oocyte. The oocyte with two pronuclei is termed zygote. A continued culture for another 24-48 hours usually results in the first few mitotic divisions of the zygote, in which case the term pre-embryos is now used. Thus, a pre-embryo consists of at least 2 cells (those two cells are called blastomers). Quite often 48-72 hours after the oocyte has been retrieved, the presence of pre-embryos is observed after the in vitro fertilisation and is followed by replacement of the pre-embryos to the woman's uterus for further development.

In one embodiment of the invention the exposure and culture of the oocytes last until zygotes and/or pre-embryos are formed. The formation of zygotes and/or pre-embryos usually occur within 16-24 hours. In a related embodiment the one or more oocytes exposed and cultured are oocytes in Metafase II (MII).

By MII is understood an oocyte which has gone through germinal vesicle break down, with the first polar body present and more or less expanded cumulus complex. These oocytes are readily recognised by the person skilled in the art of handling oocytes. Oocytes in MII are the type of oocytes prepared and ready to be fertilised by the sperm cell.

One embodiment of the present invention relates to a method for in vitro fertilisation comprising the steps of:
(a) culturing one or more GV oocytes in a culture medium, the culture medium comprising at least one meiosis activating sterol MAS, a MAS analogue, and/or an additive or additives capable of endogenous stimulation of the accumulation of at least one MAS; hereby forming one or more MII oocytes;
(b) exposing and culturing the one or more MII oocytes of step (a) with spermatozoa in a culture medium, the culture medium comprising at least one MAS, a MAS analogue, and/or an additive or additives capable of endogenous stimulation of the accumulation of at least one MAS;
   the exposure and culturing lasting at least until zygotes and/or pre-embryos are formed.

In one aspect of the invention this method is carried out with the same medium in step (a) and step (b). Hereby the natural surroundings are maintained throughout the process and a washing step is omitted. In another aspect of the invention a washing step is introduced between step (a) and step (b).

The additive or additives capable of endogenous stimulation of the accumulation of at least one MAS (e.g. FSH and EGF) may affect maturation positively and provide the cumulus-oocyte complex with a meiosis promoting effect by two different mechanisms. One may be through a positive stimulus on the cumulus cells which affect the oocyte directly. The cumulus cells may also release substances to the medium, which are able to induce resumption of meiosis and sustain further maturation of other oocytes in the medium. It is therefore yet another aspect of the present invention to improve implantation rate, the fertilisation rate, and/or the viability of the oocytes by the co-culture of several cumulus-oocyte complexes together in the same culture medium with the additive or additives. Those oocyte-cumulus complexes which, during the course of ovarian stimulation, have experienced a sub-optimal maturation or may be unable to resume meiosis in a physiological correct way, can in vitro receive a positive stimulation by the release of substances from the more mature oocyte-cumulus complexes after the stimulation by the co-culture of several oocyte-cumulus complexes together in the presence of the additive or additives. An increase in viability of co-culture and addition of the additive or additives to the culture medium will result in a more optimal maturation of those oocytes, which during the course of ovarian stimulation lack a proper stimulus to resume meiosis. As the MAS endogenously produced by the optimal matured cumulus enclosed oocytes is released to the medium, the less optimal matured oocytes will benefit from this stimulus and be induced to resume meiosis. The fertilising capacity of the retrieved oocytes will increase, and it is possible to increase the rate of fertilisation. When more viable pre-embryos are created by in vitro fertilisation in the medium with the additive or additives the likelihood of conceiving will increase in connection with infertility treatment.

The raise in content of MAS in said media with 1) naturally occurring hormones such as a glycoprotein with follicle stimulating activity (i.e. follicle stimulation hormone (FSH)), a mixture of specific FSH isoforms with an isoelectric point above 5.0, and growth factors with activity like epidermal growth factor (EGF), or with 2) substances which interfere in the biosyntetic pathway, which converts lanosterol to cholesterol in such a way that intermediates accumulate leading to an augmented MAS concentration. One aspect of the invention relates to the effect of the above mentioned substances, which increase the ovarian follicular cumulus cell production of MAS and thereby enhance the capacity of the oocyte to undergo normal pre-embryo development resulting in augmented implantation and conceptional potential of the derived pre-embryos obtained during a culture period as used in connection with assisted reproduction and infertility treatment.

It is presently contemplated that the endogenous production of MAS takes place in the cumulus cells connected to and surrounding the oocytes. Thus, in one embodiment of the invention oocytes with no or few cumulus cells are exposed and cultured with spermatozoa in a culture medium comprising MAS or a MAS analogue the implantation rate for the pre-embryo resulting from the in vitro fertilisation is increased. In another embodiment, cumulus enclosed oocytes are exposed and cultured with spermatozoa in a culture medium comprising MAS or a MAS analogue the implantation rate for the pre-embryos resulting from the in vitro fertilisation is increased.

In a preferred embodiment of the invention the culture medium comprises an additive or additives capable of endogenous stimulation of the accumulation of at least one MAS. In a related aspect of the invention, 2-30 oocytes are cultured and exposed together, such as 2-25, 2-20, or just 2-15 oocytes, wherein a few of these (such as less than 50%, e.g. less than 40%, less than 30%, or less than 20% of the oocytes) are cumulus enclosed.

Another major aspect of the invention relates to the use of a culture medium comprising at least one meiosis activating sterol (MAS), a MAS being any sterol in the metabolic pathway between lanosterol and cholesterol, a MAS analogue, and/or an additive or additives capable of endogenous stimulation of the accumulation of at least one MAS for in vitro fertilisation, the pre-embryo resulting from the in vitro fertilisation having an improved implantation rate in vivo.

Yet another embodiment relates to the use of at least one meiosis activating sterol (MAS), a MAS being any sterol in the metabolic pathway between lanosterol and cholesterol, a MAS analogue, and/or an additive or additives capable of endogenous stimulation of the accumulation of at least one MAS in cumulus enclosed oocytes for the preparation of a culture medium for in vitro fertilisation, the pre-embryo resulting from the in vitro fertilisation having an improved implantation rate in vivo.

The term meiosis activating sterol (MAS) refers to a substance which is an intermediate in the cholesterol biosynthesis between lanosterol and cholesterol (see FIG. 1). Two examples of MAS are FF-MAS (4,4-dimethyl-5α-cholesta-8,14,24-triene-3β-ol) and T-MAS (4,4-dimethyl-5α-cholest-8,24-diene-3β-ol). A MAS will preferably be a sterol. Examples of MAS compounds are mentioned in WO96/00235, WO96/27658, WO97/00884, WO98/28323, WO98/54965 and WO98/55498. It is preferred that the MAS is selected from the group consisting of FF-MAS, T-MAS, 1-methyl-zymosterol, and zymosterol.

In another embodiment a MAS analogue is added to the medium. A MAS analogue is defined as a substance causing an effect comparable to the published MAS effects. This could be the published MAS effect on oocyte maturation (Hegele-Hartung et al., 1999) or GVBD (Byskov et al., 1995) or, preferably, on implantation rate as described in example 2. More specifically, MAS and MAS analogues are compounds which in the test described in Example 7 below has a percentage germinal vesicle breakdown (hereinafter designated GVBD) which is significantly higher than the control. Preferred MAS and MAS analogues are those having a percentage GVBD of at least 50%, preferably at least 80%.

One aspect of the invention relates to the effect of the additive or additives which increase the ovarian follicular cumulus cell production of MAS and thereby enhance the capacity of the oocyte to undergo normal pre-embryo development resulting in augmented implantation and conceptional potential of the derived pre-embryos obtained during a culture period as used in connection with assisted reproduction and infertility treatment. The additive or additives are defined as the additive or additives which lead to a ratio of at least 2 between the relative content of MAS in cumulus enclosed oocytes cultured in the presence of the additive or additives, the relative content of MAS in cultured cumulus enclosed oocytes being determined by stimulating female mice with exogenous gonadotropins 48 h prior to removal of the ovaries from the mice and recovering cumulus enclosed oocytes from the ovaries by puncturing individual follicles and culturing the recovered cumulus enclosed oocytes in an α-MEM medium supplemented with 3 mg/l bovine serum albumin, 5 mg/l human serum albumin, 2 mM L-glutamin, 100 IU/ml penicillin, 100 μg/ml steptomycin, 4 mM hypoxanthine and ³H-mevalonat for 24 h at 37° C., 100% humidity and 5% CO₂ in air, followed by acidification with 50 μl 0.3M Na₂PO₄ pH=1, organic extraction three times with a five-fold surplus of n-heptane:isopropanol (3:1 v/v), purification of MAS from the organic phase by HPLC and determination of the ratio of radioactivity per cumulus enclosed oocyte between cumulus enclosed oocytes cultured in the presence of the additive or additives and cumulus enclosed oocytes cultured without the presence of the additive or additives.

It is preferred that the additive is selected from the group consisting of gonadotropins such as FSH and analogues, growth hormones such as EGF and analogues, cholesterol synthesis inhibitors such as compounds inhibiting sterol Δ14-reductase e.g. AY9944-A-7, or compounds inhibiting 4-demethylase converting T-MAS to Zymosterol, compounds activating cytochrome P450 lanosterol 14α-demethylase, and compounds with an amphotericin like effect.

The term FSH refers to proteins with follicle-stimulating activity comprising the amino acid sequences of the heterodimers of the FSH and the chains of pituitary derived proteins. However, the term FSH is also intended to refer to substances activating the FSH-receptor of the ovarian cumulus cells. Such substances are able to activate the FSH receptor located on the cumulus cells, whereby resumption of meiosis is initiated. Examples of such substances may be oligo peptides, derived from the entire sequence of the FSH molecule, or derivatives thereof.

The term EGF refers to all proteins which activate the EGF receptor (e.g. EGF and transforming-growth factor-α) and causes accumulation of MAS in the cumulus-oocyte complex. Other substances which are likely to posses similar activities include substances like activin, insulin-like growth factor I and insulin-like growth factor II.

The term cholesterol synthesis inhibitors refers to substances which interfere with the cholesterol biosynthesis in a way which results in accumulation of meiosis activating sterols. AY 9944 and Amphotericin are examples of such compounds.

It is preferred that the additive is a combination of a gonadotropin and a growth hormone, such as a combination of EGF and FSH. However, data suggest that EGF and FSH individually are capable of endogenous stimulation of the accumulation of at least one MAS. It is preferred that the concentration of EGF is between 1 and 10 ng EGF/ml, such as 9 ng/ml, 8 ng/ml, 7 ng/ml, 6 ng/ml, 5 ng/ml, 4 ng/ml, or 3 ng/ml, preferably 2 ng EGF/ml.

It is a further aspect of the present invention that the combination of EGF and FSH has a synergistic effect on the implantation rate and on oocyte maturation.

As illustrated in example 4, less and mid-acidic FSH isoforms with a pl above 5.0 are significantly more potent in inducing mouse cumulus enclosed oocytes to resume meiosis in vitro than acidic FSH isoforms. It is therefore preferred that one additive is FSH, wherein FSH is an FSH isoform with an isoelectric point above 5.0. The term FSH isoforms refers to proteins with an amino acid sequence of FSH, but which differ in their oligosaccharide structures, including the degree of terminal sialylation and/or sulfation, resulting in different isoelectric points pl's (i.e. the pH value where the net charge of the protein is zero). FSH isoforms are obtained by enzymatic or chemical modification or, preferably by chromatografocusing of naturally occurring unfractionated FSH. Carbohydrate chains are removed with such treatments without affecting the amino acid sequence. Examples of such treatments are Hydrogen Fluoride and enzymatic neuramimidase treatment resulting in partial desialylation.

FSH is derived from naturally occurring FSH such as FSH extracted from urine, or from recombinant FSH. The preferred concentration of FSH is between 2 and 200 IU FSH/I, such as between 5 and 50 IU FSH/l, e.g. 50 IU/l, 40 IU/l, 30 IU/l, 20 IU/l, or 10 IU/l, preferably 25 IU FSH/l.

One aspect of the present invention relates to a combination of additives such as FSH and EGF with a MAS or a MAS analogue. This embodiment is especially preferred when the in vitro fertilisation is performed in a medium covered with oil. Typically the medium is water soluble and the oil, e.g. mineral oil is water insoluble.

It is preferred that a simple medium consisting mainly of various salts is used as the starting point. Examples of simple media are M16, EBSS, Ham-F10, Whitten, Brinster, BWW, T6, Earle's, HTF, CZB, MTF, P1 and Menezo's B3 medium. It is well known for the person skilled in the art how media are prepared for a detailed description reference is made to Trounson and Gardner, pages 98-101.

It is also preferred that the medium comprises antibiotics (such as penicillin and/or steptomycin), and Human Serum Albumin (HSA). It is also preferred that the culture medium further comprises a pH regulatory component to maintain the pH between 7.3 and 7.5. A preferred example of a pH regulatory component is bicarbonate. It is even further preferred that the culture medium has an osmolarity of 280-300 mOsmol/kg. As will be readily acknowledged by the person skilled in the art, it is preferred that the medium also contains pyruvate.

The term culture medium reproduction refers to a medium in which in vitro fertilisation and pre-embryo culture can be performed. Consequently, the medium is able to sustain the viability of oocytes and spermatozoa in culture, facilitate fertilisation of an oocyte with a spermatozoa, and support pre-embryo development.

### REFERENCES

Baltzen M, Byskov A G (1999) Quantitation of meiosis activating sterols in human follicular fluid using HPLC and photodiode array detection. BioMedical Chromatography 13: 382-388.
Byskov A G, Yding Andersen C, Nordholm, L, Thøgersen H, Guoliang X, Wassmann O, Andersen J V Roed T. (1995). Chemical structure of novel Meiosis activating steroids crucial to reproduction. Nature, 374: 559-62.
Byskov A G, Andersen C Y, Hossaini A, Guoliang X. (1997). Cumulus cells of oocyte-cumulus complexes secrete a meiosis-activating substance when stimulated with FSH. Mol. Reprod. Dev. 46: 296-305.
Byskov A G, Baltsen M, Andersen C Y (1998) Meiosis-activating sterols: background, discovery, and possible use. J. Mol. Med., 76: 818-823.
Gòmez E., Santos, M. J. de los, Ruiz, A. et al. (1993). Effects of epidermal growth factor in the final stages of nuclear and cytoplasmic oocyte maturation in humans. Hum. Reprod., 8: 691-694.
Goud, P. T., Goud, A. P., Qian, C. et al. (1998). In-vitro maturation of human germinal vesicle stage oocytes: role of cumulus cells and epidermal growth factor in the culture medium. Hum. Reprod., 13: 1638-1644.
Hegele-Hartung C., et al., (1999) Nuclear and cytoplasmit maturation of mouse oocytes after treatment with synthetic meiosis-activating sterol in vitro. Biol. Reprod., 61: 1362-1372.
Hegele-Hartung C., et al., (1998) Oocyte maturation can be induced by a synthetic meiosis-activating sterol (MAS) leading to an improvement of IVF rate in mice. Hum. Reprod. 13:(abstract book 1), O-193.
Maruo, T., Ladines-Liave, C. A., Samoto, T. et al. (1993). Expression of Epidermal Growth Factor and its receptor in the human ovary during follicular growth and regression. Endocrinology, 132: 924-931.
Mercer E I. (1993). Inhibitors of sterol biosynthesis and their applications. Prog. Lipid. Res. 32: 357-416.
Merriman, J. A., Whittingham, D. G. and Carroll, J. (1998). The effect of follicle stimulating hormone and epidermal growth factor on the developmental capacity of in-vitro matured mouse oocytes. Hum. Reprod. 13: 690-695.
Morishige, K., Kurachi, H., Amemiya, K. et al. (1993). Menstrual stage-specific expression of epidermal growth factor and transforming growth factor-in human oviduct epithelium and their role in early embryogenesis. Endocrinology, 133: 199-207.
Salha O, Abusheika N and Sharma V (1998) Dynamics of human follicular growth and in-vitro oocyte maturation Hum. Reprod. update, 4(6): 816-832.
Schroepfer G J J, Lutsky B N, Martin J A, Huntoon S, Fourcans B, Lee W H, Vermillion J. (1972). Recent investigations on the nature of sterol intermediates in the biosynthesis of cholesterol. Proc. R. Soc. Lond. Biol. Sci., 180: 125-146.
Schramm R D and Bavister B D (1995) Effects of granulose cells and gonadotropins on meiotic and developmental competence of oocytes in vitro in non-stimulated rhesus monkeys. Hum. Reprod., 10: 887-895.
Trounson A and Gardner D K Handbook of In Vitro Fertilization, CRC press 1993.
Yoshida Y., et al., (1996) Sterol 14-demethylase P450 activity expressed in rat gonads: contribution to the formation of mammalian meiosis-activating sterol. Biochem. Biophys. Res. Comm. 223: 534-538.

### EXAMPLES

### Example 1

### Endogenous Production of Meiosis Activating Sterol in Mouse Cumulus Enclosed Oocytes Stimulated with FSH and EGF during Culture

A total of 20 immature mice were stimulated with exogenous gonadotropins (7.5 IU per mouse; Gonadoplex, Leo, Ballerup, Denmark). Forty-eight hours after injection with gonadotropins the mice were sacrificed and the ovaries removed. Oocytes were recovered by puncturing individual follicles and collected in a culture medium (α-MEM, Gibco, BRL) supplemented with 3 mg/ml bovine serum albumin, 5 mg/ml human serum albumin, 2 mM L-glutamine, 100 IU/ml penicillin, 100 μg/ml streptomycin and 4 mM hypoxanthine. A total of around 650 cumulus enclosed oocytes were isolated. The cumulus enclosed oocytes (CEO) were randomly divided into two equal pools consisting of 325 CEO each. One of the pools served as a control and was cultured in a control medium without the addition of FSH and EGF. The other served as a test and the oocytes were cultured in the presence of 25 IU/L human FSH (Gonal F, Serono Nordic, Sweden) and 2 ng/ml EGF (Sigma, USA). In addition, the test and the control culture both received radioactive labeled mevalonate (³H-mevalonat) (90 μCi ³H-mevalonat; 38 Ci/mmol: NEN Life Science Products, Boston, Mass., USA), which is a natural precursor for the production of sterols and steroids. The volume in each well was 400 μl.

After a culture period of 24 hours (37° C., 100% humidity in 5% CO₂ in air) steroids and sterols were removed from the media and the CEO, by an organic extraction after acidification with 50μ 10.3 M Na₂ PO₄, pH 1.0. A mixture of n-heptane:isopropanol (3:1 v/v) in a five-fold surplus was added and shaked vigorously for 3 hours. The organic phase was isolated and lanosterol, FF-MAS, T-MAS, cholesterol and progesterone were purified by two consecutive HPLC steps (for details see Baltzen and Byskov 1999) to involving a primary straight phase column (ChromSpher-TM Si, 5 μm, 250×4.6 mm; mobile phase: 99.5% n-heptane, 0.5% isopropanol (v/v); flow-rate: 1.0 ml/min; temp: 28° C.) and a second reverse phase column (LiChrosper-TM RP-8.5 μm, 250×4.6 mm; mobile phase: 92.5% acetonitrile, 7.5% water (v/v); flow rate: 1.0 ml/ml; temp: 40° C.). The fractions selected for further purification on the second column were dried and reconstituted in 100 μl acetonitrile. Fractions containing lanosterol, FF-MAS and T-MAS were prior to loading on the second HPLC column spiked with cold standards of the respective substances in order to avoid loss of radioactive sterols due to unspecific binding.

Radioactivity was monitored in each of the purified fractions. The biosynthesis of lanosterol, FF-MAS, T-MAS and cholesterol were quantified by measuring the incorporated radioactivity. The effect of FSH/EGF was evaluated by calculating the ratio of the radioactivity per CEO in test and control cultures.

TABLE 1
Ratio of radioactivity incorporated in sterols and steroids
between CEO cultured with and without FSH and EGF stimulation

| Lanosterol | FF-MAS | T-MAS | Cholesterol | Progesterone |
|---|---|---|---|---|
| 0.9 | 4.0 | 2.2 | 0.2 | 2.1 |

These results presented in table 1 demonstrate that the presence of FSH and EGF in cultures of CEO causes the accumulation of endogeneous produced FF-MAS and T-MAS by attenuating the conversion of T-MAS to cholesterol. FSH and EGF causes a specific effect on the accumulation of FF-MAS and T-MAS as illustrated by the fact that the ratio for lanosterol is almost unaffected and the production of cholesterol is reduced around five times. However, FSH and EGF also stimulate the production of progesterone.

### Example 2

### Significant Implantation Rate of Human Pre-Embryos Cultured in the Presence of FSH and EGF

Fifty-seven women undergoing in-vitro fertilization (IVF) treatment were inrolled in this study in order to evaluate the effect of enriching the oocyte culture medium with FSH and EGF. The following inclusion criteria were used for this study: i) age of woman below 40 years, ii) regular menstruation with intervals between 26 and 32 days, iii) normal pre-treatment levels of gonadotropins (less than 10 IU/L of FSH and LH) and iiii) more than 12 preovulatory follicles at the time of oocyte recovery. Exclusion criteria: i) cases in which intracytoplasmic sperm injection (ICSI) was used, ii) infertility caused by endocrine abnormality, e.g. polycystic ovaries. For pituitary down-regulation all women received Gonadotropin-releasing-hormone agonist (GnRHa; 0.5 mg buserelin per day) starting on cycle day 21 and lasting 10-14 days. After achievement of ovarian quiesence, stimulation with exogenous gonadotropins was initiated using a dose of 150-225 IU per day of recombinant FSH (Gonal F, Serono Nordic, Sweden) together with buserelin (0.2 mg per day). Ovulation was induced (10000 IU hCG) when more than two follicles measured more than 17 mm in diameter. Oocytes were recovered using ultrasound guided transvaginal aspiration. Half of the retrieved oocytes (every second) were cultured in a standard medium without addition of FSH and EGF, and the other half in medium supplemented with 2 ng recombinant EGF/ml and 25 IU/L of recombinant FSH (Gonal F) for the first 24 hours. The standard medium consisted of Earles Balanced Salt Solution, supplemented with sodium bicarbonate (NaHCO₃ 2.1 g/L), pyruvate (11 mg/L), Human Serum Albumin (10 mg/ml), and penicillin (100 IU/L). Before use, the medium is adjusted to 280 mOsmol/kg and to pH 7.3-7.4 and sterile filtered. After 24 hours during which the in vitro fertilisation takes place, the medium was removed and replaced with a standard medium which were similar in both groups. The culture was continued for 48 hours (to a total of 72 hours), after which replacement of pre-embryos was performed. Thereby, each woman served as her own control. The morphological best looking pre-embryo over-all decided the group from which the pre-embryos for transfer were selected. A maximum of two pre-embryos were replaced, representing the over-all best looking pre-embryo plus next best looking pre-embryo from that group. The criteria for selecting the morphological best looking pre-embryo was done according to Yding Andersen et al. (1991). Consequently, pre-embryos from the two groups were not mixed making it possible later unequivocally to decide from which group a pregnancy resulted.

Out of the 57 women three had oocytes which did not cleave in vitro and three had—due to the possible development of hyper-stimulation syndrome—all developed pre-embryos cryopreserved.

TABLE 2
Patient characteristics and results of ovarian
stimulation of women undergoing IVF treatment

| | Control medium | FSH/EGF medium |
|---|---|---|
| No. of women | 24 | 27 |
| Age (years) | 31.4 (24-39) | 30.7 (26-37) |
| Pre-treatment FSH levels (IU/l) | 5.5 ± 0.3 | 6.5 ± 0.4 |
| Pre-treatment LH levels (IU/l) | 5.7 ± 0.6 | 4.9 ± 0.3 |
| Fertility diagnosis: | | |
| Tubal factor | 15 (63%) | 18 (68%) |
| Endometriosis | 6 (25%) | 4 (15%) |
| Idiopathic | 3 (12%) | 5 (18%) |
| No. of stimulation days | 9.9 ± 0.2 | 9.4 ± 0.2 |
| Exogenous FSH (IU) | 1800 ± 90 | 1725 ± 90 |
| Oestradiol cycle day 2 (pmol/l) | 115 ± 15 | 112 ± 12 |
| Oestradiol cycle day 9 (pmol/l) | 2980 ± 450 | 3860 ± 500 |

TABLE 3
Outcome of IVF treatment and implantation
rate of the derived pre-embryos.

| | Control | |
|---|---|---|
| | medium | FSH/EGF medium |
| No. of women | 24 | 27 |
| No. of oocytes | 448 | 456 |
| No. of fertilized oocytes | 327 (73%) | 356 (78%) NS |
| No. of pre-embryos | 315 (70%) | 338 (74%) NS |
| No. oocytes with ≧3 proneuclei | 23 (5.1%) | 24 (5.3%) |
| No. of pre-embryo replacements | 24 | 27 |
| No. of pre-embryos replaced | 45 | 52 |
| No. of positive pregnancy tests | 6 (25%) | 15 (56%) p < 0.03 |
| No. of clinical pregnancies | 6 (25%) | 13 (48%) NS |
| Implantation rate | 6 (13%) | 18 (38%) p < 0.025 |

None of the above monitored parameters, presented in tables 2 and 3 differ significantly between the two groups except for the implantation rate and the number of positive pregnancy tests, which is significantly higher in the group of oocytes/pre-embryos exposed to a medium containing FSH and EGF compared to that of the control.

The culture medium from 40 oocytes in each of the two groups were collected and pooled to make up one pool from the control medium and one pool from the medium enriched with FSH and EGF. The medium was collected directly from the four-well dishes using a pasteur-pipette with manual suction. The steroids and sterols in the two pools were extracted as described in Example 1.

TABLE 3a
Quantification of MAS in culture medium in which oocytes have
grown with or without enrichment of FSH and EGF

| | Lanosterol | FF-MAS | T-MAS | FF + T-MAS | Cholesterol | Progesterone |
|---|---|---|---|---|---|---|
| | (ng) | (ng) | (ng) | (ng) | (μg) | (μg) |
| Control | <7 | <4 | <7 | <11 | 15 | 1.7 |
| medium | | | | | | |
| FSH/EGF | 90 | 117 | 94 | 210 | 13 | 5.4 |
| medium | | | | | | |

The results given in table 3a show that the presence of FSH and EGF causes an accumulation of FF-MAS and T-MAS, together with their precursor lanosterol, contrasting the control medium where these substances cannot be detected. The concentration of cholesterol remain unchanged, whereas the progesterone content also seems to increase in the medium enriched with FSH and EGF.

### Example 3

### Effect of FSH and EGF on Resumption of Meiosis of Mouse Oocytes Cultured In Vitro

Immature female mice (B6D2-F1 strain C57B1/2J) were kept under controlled light and temperature conditions with free access to food and water. Ovarian stimulation was performed when the mice weighed 10-16 grams and consisted of an intra-peritoneal injection of Gonadoplex (Leo, Copenhagen, Denmark) containing (7.5 U/mouse). The animals were killed by cervical dislocation 44-48 h later. The media used for the culture of oocytes consisted of α-Minimum Essential Medium (α-MEM), with Earles Balanced Salt Solution (EBSS), 4 mM hypoxantine (HX), 3 mg/ml Bovine Serum Albumin, 0.23 mM pyruvate, 2 mM glutamine, 100 IU/ml penicillin and 100 mg/ml streptomycin. The ovaries were recovered and placed in HX-medium where an initial cleaning and removal of connective tissue was performed. Oocytes were isolated from the ovaries by puncturing individual follicles using 25 gauge needles. Isolation of oocytes were performed in HX-medium to prevent resumption of meiosis until the tests were ready to be performed. The oocytes were then washed 3 times in control medium before the start of each experiment. Cumulus enclosed oocytes were cultured separately in 4-well dishes (Nuncleon, Roskilde, Denmark), 0.4 ml medium in each well containing control medium or medium supplemented with FSH and/or EGF addition in a 100% humidified atmosphere of 5% CO₂ with 95% air at 37° C. The culture period was 22-24 hours. By the end of the culture period, germinal vehicle breakdown (GVBD) was scored by examining the oocyte in an inverted microscope. The percentage of oocytes with GVBD per total number of oocytes (% GVBD) was calculated.

TABLE 4
Effect of FSH and EGF on resumption of meiosis
in mouse oocytes cultured in vitro

| | No. of oocytes | No. of GVBD | % GVBD |
|---|---|---|---|
| Control medium | 154 | 29 | 198^{a,b,c} |
| FSH (10 IU/l) | 162 | 73 | 45^{b} |
| EGF (0.5 ng/ml) | 68 | 21 | 31^{c,d} |
| FSH (10 IU/l) + EGF | 63 | 33 | 52^{a,d} |
| (0.5 ng/ml) | | | |

Values with similar letter differ significantly P < 0.05

The results presented in table 4 demonstrate that FSH and EGF enhance oocyte maturation of mouse oocytes in vitro and that a combination of FSH and EGF seem to give an enhanced effect.

### Example 4

### Effect of Different FSH Isoforms on Resumption of Meiosis of Mouse Oocytes Cultured In Vitro

The experiments were performed similar to those described in example 3.

FSH isoforms were isolated from human pituitary extracts using glycoprotein extraction followed by chromatofocusing. Before being applied to induce resumption of meiosis in mouse oocytes, the FSH isoforms were thoroughly dialysed against the control medium.

Each FSH isoform fraction were tested in serial dilutions and the dilution at which 50% of the oocytes resumed meiosis was determined (equivalent dose of 50% GVBD (ED_{50%} GVBD), the ED_{50%} GVBD value, and determination was repeated 3 to 5 times for each FSH isoform fraction. In each 4-well dish, one well always served as a control and contained oocytes cultured in the control medium without FSH. Each well contained between 30 and 40 CEO.

The FSH concentration of the isoform fractions tested was monitored using two methods: 1) a radio-immuno-assay employing specific rabbit anti-FSH antibodies and ¹²⁵I-labelled human FSH as a tracer. Bound and free radioactivity was separated by anti-rabbit immunoglobulin coated to dextran particles. Inter and intra-assay variation of a sample containing 25 IU/l were 7% and 5% respectively, 2) a Chinese hamster ovary cell (CHO-cell) line which stably expresses the recombinant human FSH receptor and which upon stimulation with FSH releases cAMP. The amount of cAMP generated by the FSH isoform fractions was related to a standard. The inter- and intra-assay variation was 15% and 18% respectively.

TABLE 5
Effect of FSH isoform fractions on resumption
of meiosis in mouse oocytes

| | Total | Mean no. | | ED_{50%} GVBD |
|---|---|---|---|---|
| FSH isoform | no. of | of oocytes | ED_{50%} GVBD | CHO-cell assay |
| fraction | oocytes | per well | RIA (IU/l) | (ng/l) |
| Less acidic | 517 | 34 | 6.4 ± 0.32^{a} | 142 ± 7^{c} |
| (pl 6.43-5.69) | | | | |
| Mid acidic | 1088 | 37 | 6.1 ± 0.67^{a} | 146 ± 16^{c} |
| (pl 5.62-4.96) | | | | |
| Acidic | 608 | 34 | 12.2 ± 0.74^{b} | 215 ± 13^{d} |
| (pl 4.69-3.75) | | | | |

Values are mean ± SD.
The ED_{50%} GVBD value is the concentration of FSH were 50% of the oocytes have entered meiosis after the 24 h culture period.
Within each column values with a similar letter are not significantly different and values with different letters show the following levels of significance:
^{a,b}p < 0.0005;
^{c,d}p < 0.001.

The results demonstrated in table 5 indicate that less and mid-acidic FSH isoforms with a pl above 5.0 are significantly more potent in inducing mouse CEO's to resume meiosis in vitro than acidic FSH isoforms. Almost the double concentration of acidic FSH isoforms was required to induce 50% of the oocytes to resume meiosis compared to that of less and mid-acidic isoforms.

The actual levels of FSH isoform needed to induce 50 percent of the oocytes to resume meiosis obtained in this study compare favourably to studies in which unfractionated FSH was used, where the ED_{50%} GVBD value was around 8 IU/l.

Whereas the mid-cycle of FSH in circulation reaches an amplitude of around 10-15 IU/l, the intrafollicular level of FSH which most likely represent the concentration to which the CEO is exposed to in vivo has in preovulatory follicular fluid from women just before ovulation been measured to 4-6 IU/l. These data compare favourably to the effective doses of especially the less and mid-acidic isoforms in the mouse system, in which the oocytes are exposed to FSH for 20 to 24 hours.

As described in example 2, unfractionated FSH in a concentration of 25 IU/l was used in order to secure an optimal stimulation of the human oocytes, also those oocytes which in vivo received a sub-optimal maturation or those which were immature at the time of retrieval, which could be expected to be less responsive to the FSH stimulus.

### Example 5

### Induction of Resumption of Meiosis and Endogenous Production of Meiosis Activating Sterol in Mouse Cumulus Enclosed Oocytes Exposed to an Inhibitor of Sterol Δ14Reductase (AY9944-A-7) during Culture

Immature female C57BI/2J B6D2 mice (11-15 g) were stimulated with exogenous gonadotropins (7.5 IU per mouse; Gonadoplex, Leo, Ballerup, Denmark). Forty-eight hours after injection with gonadotropins, the mice were sacrificed and the ovaries removed. Cumulus enclosed oocytes (CEO) were recovered by puncturing individual follicles and collected in a culture medium (a-MEM, Gibco, BRL) supplemented with 3 mg/ml bovine serum albumin, 5 mg/ml human serum albumin, 2 mM L-glutamine, 100 IU/ml penicillin, 100 μg/ml streptomycin and 4 mM hypoxanthine (control medium).

Induction of resumption of meiosis in mouse cumulus enclosed oocytes exposed to an inhibitor of sterol Δ14-reductase (AY9944-A-7) during culture.

Oocytes from 10-15 mice were pooled and randomly divided into the different test groups. The tests were performed using 4-well dishes (Nunclon, Roskilde, Denmark) each well containing 20-50 oocytes in 400 μl culture medium. Each dish had one well serving as control and the three other wells as tests.

All test media were prepared using HX-medium. AY9944-A-7 (AY) was kindly provided by Wyeth-Ayerst, Princeton, N.J., USA. AY has no structural similarities to MAS. Stock solutions of AY9944 in water were stored at −20° C., and was added directly to the HX-medium immediately prior to oocyte culture. AY was used in concentrations from 0.2 to 25 μM.

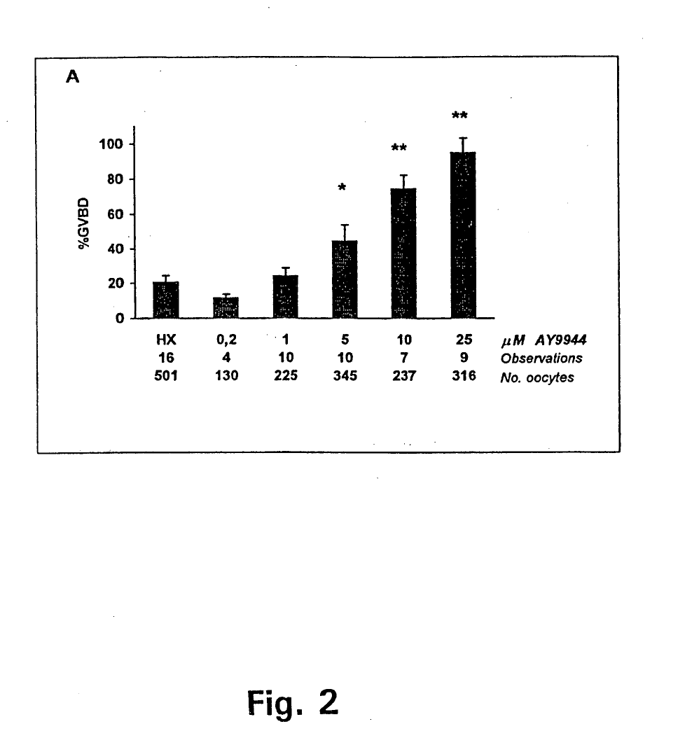

The results presented in FIG. 2 show that AY stimulates oocyte maturation of CEO in a dose-dependent way. Concentrations of 5, 10 and 25 μM increase GVBD significantly compared to the controls (p<0.05, p<0.001 and p<0.001, respectively).

Endogenous production of Meiosis Activating Sterol in mouse cumulus enclosed oocytes exposed to an inhibitor of sterol A14-reductase (AY9944-A-7) during culture.

Cumulus enclosed oocytes (CEO) were isolated and randomly divided into two equal pools consisting of 250 CEO each. One of the pools served as a control and was cultured in a control medium without the addition of AY. The other served as a test and the oocytes were cultured in the presence of 10 μM AY. In addition, the test and the control culture both received radioactive labeled mevalonate (³H-mevalonat) (90 μCi ³H-mevalonat; 38 Ci/mmol: NEN Life Science Products, Boston, Mass., USA), which is a natural precursor for the production of sterols and steroids. The volume in each well was 400 μl.

After a culture period of 24 hours, the steroids and sterols were extracted as described in Example 1.

The biosynthesis of lanosterol, FF-MAS, T-MAS and cholesterol were quantified by measuring the incorporated radioactivity. The effect of AY was evaluated by calculating the ratio of the radioactivity per CEO in test and control cultures.

TABLE 6
Ratio of radioactivity incorporated in sterols and steroids obtained from
CEO cultured with and without exposure to AY during the culture

| Lanosterol | FF-MAS | T-MAS | Cholesterol | Progesterone |
|---|---|---|---|---|
| 2.3:1 | 11:1 | 1:5.2 | 1:44 | 2.8:1 |

These results demonstrate that the presence of AY in cultures of CEO causes the accumulation of endogenous produced FF-MAS and T-MAS by attenuating the conversion of T-MAS to cholesterol. AY causes a specific effect on accumulation of FF-MAS as illustrated by the fact that Lanosterol is only accumulated to a small extend, whereas the production of T-MAS and cholesterol is reduced around 5 and 44 times, respectively.

### Example 6

### Resumption of Oocyte Meiosis in Mouse Cumulus Enclosed Oocytes Exposed to a Cholesterol Biosynthesis Inhibitor, Amphotericin B, during Culture

Amphotericin B is a medical product used against fungal infections by inhibiting cholesterol biosynthesis. It has no structural similarities to MAS. Its effect on resumption of meiosis was tested on mouse cumulus enclosed oocytes, in vitro. Ovarian stimulation was performed on immature female mice and the cumulus enclosed oocytes (CEO) isolated as described in Example 3.

Cumulus enclosed oocytes were cultured separately in 4-well dishes (Nuncleon, Roskilde, Denmark), 0.4 ml medium in each well containing control medium or medium supplemented with Amphotericin B (Bristol-Myers Squibb) in a 100% humidified atmosphere of 5% CO₂ with 95% air at 37° C.

The effect of Amphotericin in concentrations of 0.6 and 1.2 μg/ml was evaluated after 22 hours in culture.

In another series of experiments, i.e. priming of CEO, the culture period with Amphotericin, priming period, varied from 5 min. to 240 min. in test medium with 1.2 μg/ml Amphotericin. After the priming period, the CEO were transferred to control medium and the culture continued for a total of 22 hours.

By the end of the culture period, germinal vehicle breakdown (GVBD) was scored by examining the oocyte in an inverted microscope. The percentage of oocytes with GVBD per total number of oocytes (% GVBD) was calculated.

TABLE 7
Effect of amfotericin on resumption of meiosis
in mouse cumulus enclosed oocytes in vitro

| Amfotericin (μg/ml) | No. of oocytes | No. of GVBD | % GVBD |
|---|---|---|---|
| Control | 176 | 28 | 16 |
| 0.6 | 94 | 26 | 28 |
| 1.2 | 121 | 92 | 76 |

TABLE 8
Effect of amfotericin (1.2 μg/ml) priming (i.e. the period
in which the CEO's are exposed) on resumption of
meiosis in mouse cumulus enclosed oocytes in vitro

| Time (minutes) | No. of oocytes | No. of GVBD | % GVBD |
|---|---|---|---|
| Control | 175 | 26 | 15 |
| 5 | 98 | 21 | 21 |
| 10 | 88 | 26 | 29 |
| 30 | 116 | 42 | 36 |
| 120 | 50 | 25 | 50 |
| 240 | 49 | 34 | 69 |

The results presented in tables 7 and 8 demonstrate that Amphotericin causes a dose dependent resumption of oocyte meiosis.

### Example 7

### Method used for Electing MAS and MAS Analogues

Oocytes were obtained from immature female mice (C57BL/6J x DBA/2J F1, Bomholtgaard, Denmark) weighing 13-16 grams, that were kept under controlled temperature (20-22° C.), light (lights on 06.00-18.00) and relative humidity (50-70%). The mice received an intra-peritoneal injection of 0.2 ml gonadotropins (Gonal-F, Serono) containing 20 IU FSH and 48 hours later the animals were killed by cervical dislocation. The ovaries were dissected out and the oocytes were isolated in Hx-medium (see below) under a stereomicroscope by manual rupture of the follicles using a pair of 27 gauge needles. Spherical oocytes displaying an intact germinal vesicle (hereinafter designated GV) were divided in cumulus enclosed oocytes (hereinafter designated CEO) and naked oocytes (hereinafter designated NO) and placed in α-minimum essential medium (α-MEM without ribonucleosides, Gibco BRL, Cat. No. 22561) supplemented with 3 mg/ml bovine serum albumin (BSA, Sigma Cat. No. A-7030), 5 mg/ml human serum albumin (HSA, Statens Seruminstitut, Denmark), 0.23 mM pyruvate (Sigma, Cat. No S-8636), 2 mM glutamine (Flow Cat. No. 16-801), 100 IU/ml penicillin and 100 mg/ml streptomycin (Flow, Cat No. 16-700). This medium was supplemented with 3 mM hypoxanthine (Sigma Cat. No. H-9377) and designated Hx-medium. The oocytes were rinsed three times in Hx-medium and oocytes of uniform size were divided into groups of CEO and NO. CEO and NO were cultured in 4-well multidishes (Nunclon, Denmark) in which each well contained 0.4 ml of Hx-medium and the compound to be tested in a concentration of 10 mM. One control well (i.e., 35-45 oocytes cultured in identical medium with no addition of test compound) was always cultured simultaneously with 3 test wells (35-45 oocytes per well supplemented with test compound). The oocytes were cultured in a humidified atmosphere of 5% CO₂ in air for 24 hours at 37° C. By the end of the culture period, the number of oocytes with GV, GVB and polar bodies (hereinafter designated PB), respectively, were counted using a stereo microscope (Wildt, Leica MZ 12). The percentage of GVB, defined as percentage of oocytes undergoing GVB per total number of oocytes in that well, was calculated as: % GVB=((number of GVB+number of PB)/total number of oocytes)×100.

1-18. (canceled)

19. A method for in vitro fertilisation comprising the step of: exposing and culturing one or more MII oocytes with spermatozoa in a culture medium, the culture medium comprising at least one mas, a mas analogue, or an additive or additives capable of endogenous stimulation of the accumulation of at least one MAS; the exposure and culturing lasting until zygotes and/or pre-embryos are formed.

20. (canceled)

21. A method according to claim 19, wherein the MII oocytes are cumulus enclosed oocytes.

22. (canceled)

23. A method according to claim 19, wherein MAS is selected from the group consisting of FF-MAS, T-MAS, 1-methylzymosterol and zymosterol or mixtures thereof.

24. A method according to claim 19, wherein MAS is FF-MAS.

25. A method according to claim 19, wherein MAS is T-MAS.

26. A method according to claim 19, wherein the culture medium comprises a MAS analogue.

27. A method according to claim 19, wherein the oocytes are nude.

28. A method according to claim 19, wherein the culture medium comprises at least one MAS or at least one MAS analogue.

29. A method according to claim 19, wherein 1-15 oocytes are cultured and exposed together.

30. A method according to claim 19, wherein the culture medium comprises an additive or additives capable of endogenous stimulation of the accumulation of at least one MAS.

31. The method according to claim 21, wherein the additive or additives leads to a ratio of at least 2 between the relative content of MAS in cumulus enclosed oocytes cultured in the presence of the additive or additives, the relative content of MAS in cultured cumulus enclosed oocytes being determined by stimulating female mice with exogenous gonadotropins 48 h prior to removal of the ovaries from the mice and recovering cumulus enclosed oocytes from the ovaries by puncturing individual follicles and culturing the recovered cumulus enclosed oocytes in an α-MEM medium supplemented with 3 mg/l bovine serum albumin, 5 mg/l human serum albumin, 2 mM L-glutamine, 100 IU/ml penicillin, 100 μg/ml steptomycin, 4 mM hypoxanthine and ³H-mevalonat for 24 h at 37° C., 100% humidity and 5% CO₂ in air, followed acidification with 50 μl 0.3M Na₂PO₄ pH=1, organic extraction three times with a five-fold surplus of n-heptane:isopropanol (3:1 v/v), purification of MAS from the organic phase by HPLC and determination of the ratio of radioactivity per cumulus enclosed oocyte between cumulus enclosed oocytes cultured in the presence of the additive or additives and cumulus enclosed oocytes cultured without the presence of the additive or additives.

32. The method according to claim 19, wherein the additive is a combination of a gonadotropin and a growth hormone.

33. The method according to claim 19, wherein the additive is a combination of EGF and FSH.

34. The method according to claim 19, wherein the additive is EGF.

35. The method according to claim 19, wherein the additive is FSH.

36. The method according to claim 35, wherein FSH is an FSH isoform with an isoelectric point above 5.0.

37. The method according to claim 35, wherein the FSH is derived from naturally occurring FSH.

38. The method according to claim 35, wherein FSH is derived from FSH extracted from urine, or from recombinant FSH.

39. The method according to claim 19, wherein the culture medium comprises FSH at a concentration between 2 and 200 IU FSH/l.

40. The method according to claim 19, wherein the culture medium comprises EGF at a concentration between 1 and 10 ng EGF/ml.

41. The method according to claim 19, wherein the additive is amphotericin.

42. The method according to claim 19, wherein the in vitro fertilisation is in vitro fertilisation of human oocytes.

43. A method for in vitro fertilisation comprising the steps of: (a) one or more GV oocytes in a culture medium, the culture medium comprising at least one MAS, a MAS analogue, or an additive or additives capable of endogenous stimulation of the accumulation of at least one MAS; hereby forming one or more oocytes; (b) exposing and the one or more MII oocytes of step (a) with spermatozoa in a culture medium, the culture medium comprising at least one MAS, a MAS analogue, or an additive or additives capable of endogenous stimulation of the accumulation of at least one MAS; the exposure and lasting until zygotes and/or pre-embryos are formed.
